(19) United States
(12) Patent Application Publication
(10) Pub. No.: US 2004/0241204 A1
(43) Pub. Date: Dec. 2, 2004

(54) Sustained release pharmaceutical composition

(76) Inventors: Serge R. Martinod, Groton, CT (US); Malcolm Brandon, Bulleen (AU)

Correspondence Address: MORGAN LEWIS & BOCKIUS LLP, 1111 PENNSYLVANIA AVENUE NW, WASHINGTON, DC, 20004, US

(21) Appl. No.: 10/482,336

(22) PCT Filed: Jul. 1, 2002
(86) PCT No.: PCT/AU02/00865

(30) Foreign Application Priority Data
Jun. 29, 2001 (AU) PR 6025

(51) Int. Cl. A61F 2/00

(52) U.S. Cl. 424/426

(57) ABSTRACT

A sustained release apparatus including a plurality of sustained release mini-implants or pellets; each mini-implant including a sustained release support material; and a pharmaceutically active composition carried in or on the sustained release support material; the pharmaceutically active composition including at least one pharmaceutically active component; and a carrier therefor; each implant being of insufficient size and/or payload individually to provide a predetermined desired threshold blood level of pharmaceutical active for treatment of a selected indication.

The present invention relates to a sustained release pharmaceutical composition, and in particular a sustained release composition in an implant or pellet form. More specifically, the present invention relates to a sustained release pharmaceutical composition which provides a significant increase in the rate of release of the pharmaceutical agent.

A number of drug delivery systems are known in the prior art.

For example, a controlled drug-release preparation using as a carrier a hydrophobic polymer material, which is non-degradable after administration into the living body. There are two methods of controlling release of a drug from such preparation; one, using an additive such as an albumin (Japanese patent publication (Tokkohei) No. 61959/1995), and another, by forming an outer layer consisting of hydrophobic polymer alone (Japanese patent publication (Tokkohei) No. 187994/1995).

However, where a disease indication requires the achievement of a high threshold blood plasma level and/or requires the delivery of multiple pharmaceuticals and/or requires sustained release to be continued over an extended period at high levels, the drug delivery systems known in the prior art generally exhibit insufficient drug carrying capacity and release rate that are too slow to achieve high blood levels over a sustained time period.

Whilst it is theoretically possible to increase the amount of active delivered by increasing the size of the drug delivery systems in one or more dimensions (e.g. length or diameter), this may not achieve the anticipated result, e.g. as this may lead to “dose dumping” which may be harmful or even lethal to the animal to be treated. Alternatively the large size of the apparatus may prevent its use even with relatively large animals, in particular cattle.

For example, such drug delivery implants may be placed subcutaneously in the ear of an animal. This may be physically impossible where the size of the implant becomes too large.

Further, it has been found that use of multiple implants does not provide the required threshold blood level of pharmaceutical required to successfully treat the disease indication to be treated. This also is limiting due to the total bulk of the implants used.

It is, accordingly, an object of the present invention to overcome or at least alleviate one or more of the difficulties and deficiencies related to the prior art.

In a first aspect of the present invention, there is provided a sustained release apparatus including a plurality of sustained release mini-implants or pellets;

each implant including

a sustained release support material; and

a pharmaceutically active composition carried in or on the sustained release support material;

the pharmaceutically active composition including

at least one pharmaceutically active component; and

a carrier therefor;

each implant being of insufficient size and/or payload individually to provide a predetermined desired threshold blood level of pharmaceutical active for treatment of a selected indication.

Applicants have surprisingly found that the threshold blood level of a pharmaceutical active required to treat a particular, e.g. disease, indication may be achieved utilising a series of mini-implants or pellets which individually are of little or no value in treating the indication.

Preferably the sustained release apparatus may provide approximately zero order release of pharmaceutical active.

Preferably the plurality of sustained release mini-implants or pellets in combination may provide a blood level of pharmaceutical active at least equal to a predetermined threshold for an extended period, e.g. of approximately 1 to 24, preferably 1 to 4 weeks for an ivermectin active.

In one embodiment, the plurality of sustained release mini-implants or pellets may be of two or more different sizes such that they provide a blood level of pharmaceutical active of approximately 1.25 to 3 times the desired threshold blood level for an extended, though relatively short, time period, e.g. of approximately 1 to 4 weeks, and also provide a blood level of pharmaceutical active at or near the desired threshold blood level over a longer time period, e.g. of approximately 4 to 52 weeks.

In a further preferred embodiment, there is provided a sustained release kit including

a plurality of sustained release mini-implants or pellets packaged for delivery in a single treatment;

each mini-implant or pellet including

a sustained release support material; and

a pharmaceutically active composition carried in or on the sustained release support material;

the pharmaceutically active composition including

at least one pharmaceutically active component; and

a carrier therefor;

each implant being of insufficient size individually to provide a predetermined desired threshold blood level of pharmaceutical active for treatment of a selected indication.

Preferably the mini implants or pellets are provided in at least two different sizes, as discussed above.

More preferably, the mini-implants or pellets are provided

in a first size which provides a blood level of pharmaceutical active of approximately 1.25 to 3 times the desired threshold blood level for a first relatively short time period; and

in a second size which provides a blood level of pharmaceutical active at or near the desired threshold blood level for a second longer time period.

In a further preferred embodiment, each mini-implant includes

a pharmaceutical active-containing inner layer; and

a water-impermeable outer layer.

Optionally the sustained release kit further includes a sustained release delivery apparatus. For example, in veterinary applications, an injector instrument for subcutaneous delivery of standard size pellets may be used as the sustained release delivery apparatus.

The multiple mini-pellets may be provided in a single cartridge for use in a standard injector instrument which in turn disperse as individual mini-pellets within the body of the animal to be treated.

In a further preferred form of the present invention, the plurality of sustained release implants may be provided in a biodegradable sheath. The biodegradable sheath may be formed of a water-soluble material.

The water-soluble material utilised in the biodegradable sheath may be selected from one or more of the water-soluble substances described below.

Each sustained release mini-pellet according to the present invention may be biodegradable.

Each sustained release mini-pellet according to the present invention may be of the covered rod or matrix type. A covered rod-like shape is preferred.

For example each sustained release mini-pellet may be approximately 0.1 to 0.5 times, preferably approximately 0.20 to 0.40 times, the length of a single rod shaped implant, and capable of providing the desired threshold blood level, depending on the pharmaceutical active selected.

For example, in veterinary applications, a typical cattle implant is the product sold under the trade designation “Revalor”, and containing as pharmaceutical actives trembolone acetate and estradiol. This implant has the dimensions 4 mm×4 mm. The equivalent implant according to the present invention may have dimensions of 4 mm×2 mm.

In humans, a typical implant is the product sold under the trade designation “Norplant” and containing levonorgestrel as active. The implant has the dimensions 02.4 mm in diameter and 34 mm in length. The equivalent implant according to the present invention may have the dimensions of 2.4 mm×10 mm.

As discussed above, the mini-pellet or implants may exhibit two or more different sizes. In general, the longer the mini-implant, the longer the maintenance of sustained release, but the lower maximum the blood level of active achieved.

The sustained release delivery apparatus may take the form of a covered rod or dispersed matrix structure. Such a multi mini-pellet system permits the treatment of diseases over an extended period with pharmaceutically active components which have heretofore not been applicable to such diseases as it has not been possible to achieve the required threshold blood plasma levels to be efficacious and to maintain those blood levels over an extended period of time.

Preferably the sustained release delivery apparatus may provide approximately zero order release of pharmaceutical active.

For example, in veterinary applications, the pharmaceutically active component ivermectin is a mixture of not less than 90% ivermectin H₂B₁a and not more than 5% ivermectin H₂B₁b having the respective molecular weights 875.10 and 861.07. ivermectin is a potent macrocyclic lactone disaccharide antiparasitic agent used to prevent and treat parasite infestations in animals. The compound has activity against both internal and external parasites as well as being effective against arthropods, insects, nematodes, filarioidea, platyhelminths and protozoa.

The sustained release support material may take the form of a support matrix or rod, preferably a covered rod structure. The sustained release support material may take the form of an open ended cylindrical rod.

The sustained release support material may be formed from a biodegradable or biocompatible material, preferably a biocompatible hydrophobic material. The biocompatible material may be selected from the group consisting of polyesters, polyamino acids, silicones, ethylene-vinyl acetate copolymers and polyvinyl alcohols. Preferably the sustained release support material is a silicone material. A silicone rod is preferred. The silicone material may be a porous silicon or Biosilicon material, for example as described in international patent application PCT/GB99/01185, the entire disclosure of which is incorporated herein by reference. A mesoporous, microporous or polycrystalline silicon or mixtures thereof may be used.

Biodegradable polymers that may be employed in the present invention may be exemplified by, but not limited to, polyesters such as poly(lactic acid-glycolic acid) copolymers (PLGA), hydrophobic polyamino acids such as polyaranin, polyleucine, polyanhydride, poly(glyceol-sebacate)(PGS), Biopol, and the like. The hydrophobic polyamino acids mean polymers prepared from hydrophobic amino acids.

Nonbiodegradable polymers that may be employed in the present invention may be exemplified by, but not limited to, silicones, polytetrafluoroethylenes, polyethylenes, polypropylenes, polyurethanes, polyacrylates, polymethacrylates such as polymethylmethacrylates, etc., ethylene-vinyl acetate copolymers, and others.

More preferably a silicone elastomer as described in copending Australian provisional patent application PR7614, to applicants (the entire disclosure of which is incorporated herein by reference), may be used. For example the silicon elastomer may be formed from a methyl-vinyl siloxane polymer including a fumed silica as reinforcing filler.

The pharmaceutically active composition, as described above, includes at least one pharmaceutically active component. The pharmaceutically active component may be exemplified by, but not limited to, one or more selected from the group consisting of:

| | |
|---|---|
| Acetonemia preparations | Anabolic agents |
| Anaesthetics | Analgesics |
| Anti-acid agents | Anti-arthritic agents |
| Antibodies | Anti-convulsivants |
| Anti-fungals | Anti-histamines |
| Anti-infectives | Anti-inflammatories |
| Anti-microbials | Anti-parasitic agents |
| Anti-protozoals | Anti-ulcer agents |
| Antiviral pharmaceuticals | Behaviour modification drugs |
| Biologicals | Blood and blood substitutes |
| Bronchodilators and expectorants | Cancer therapy and related |
| | pharmaceuticals |
| Cardiovascular pharmaceuticals | Central nervous system |
| | pharmaceuticals |
| Coccidiostats and coccidiocidals | Contraceptives |
| Contrast agents | Diabetes therapies |
| Diuretics | Fertility pharmaceuticals |
| Growth hormones | Growth promoters |
| Hematinics | Hemostatics |
| Hormone replacement therapies | Hormones and analogs |
| Immunostimulants | Minerals |
| Muscle relaxants | Natural products |
| Nutraceuticals and nutritionals | Obesity therapeutics |
| Ophthalmic pharmaceuticals | Osteoporosis drugs |
| Pain therapeutics | Peptides and polypeptides |
| Respiratory pharmaceuticals | Sedatives and tranquilizers |
| Transplantation products | Urinary acidifiers |
| Vaccines and adjuvants | Vitamins |

The pharmaceutically active component may include a water-insoluble pharmaceutical, a water-soluble pharmaceutical or mixtures thereof.

The water-soluble pharmaceutical actives useful in the sustained release delivery apparatus according to the present invention include such drugs as peptides, polypeptides, proteins, glycoproteins, polysaccharides, and nucleic acids.

The present invention is particularly appropriate for pharmaceuticals that are very active even in extremely small quantities and whose sustained long-term administration is sought. When used in substantially increased quantities, such pharmaceuticals may be applied to disease indications heretofore untreatable over an extended period. The pharmaceuticals may be exemplified by, but not limited to, one or more selected from the group consisting of cytokines (eg. interferons and interleukins), hematopoletic factors (eg. colony-stimulating factors and erythropoietin), hormones (eg. growth hormone, growth hormone releasing factor, calcitonin, leuteinizing hormone, leuteinizing hormone releasing hormone, and insulin), growth factors (eg. somatomedin, nerve growth factor), neurotrophic factors, fibroblast growth factor, and hepatocyte proliferation factor; cell adhesion factors; immunosuppressants; enzymes (eg. asparaginase, superoxide dismutase, tissue plasminogen activating factor, urokinase, and prourokinase), blood coagulating factors (eg. blood coagulating factor VIII), proteins involved in bone metabolism (eg. BMP (bone morphogenetic protein)), and antibodies.

The interferons may include alpha, beta, gamma, or any other interferons or any combination thereof. Likewise, the interleukin may be IL-1, IL-2, IL-3, or any others, and the colony-stimulating factor may be multi-CSF (multipotential CSF), GM-CSF (granulocyte-macrophage CSF), G-CSF (granulocyte CSF), M-CSF (macrophage CSF), or any others.

Vaccines are particularly preferred. The vaccines useful in the sustained release delivery apparatus according to the present invention may be exemplified by, but not limited to, one or more selected from the group consisting of

| | |
|---|---|
| Adenovirus | Anthrax |
| BCG | Chlamydia |
| Cholera | Circovirus |
| Classical swine fever | Coronavirus |
| Diphtheria-Tetanus (DT for children) | Diphtheria-Tetanus (tD for adults) |
| Distemper virus | DTaP |
| DTP | E coli |
| Eimeria (coccidosis) | Feline immunodeficiency virus |
| Feline leukemia virus | Foot and mouth disease |
| Hemophilus | Hepatitis A |
| Hepatitis B | Hepatitis B/Hib |
| Herpes virus | Hib |
| Influenza | Japanese Encephalitis |
| Lyme disease | Measles |
| Measles-Rubella | Meningococcal |
| MMR | Mumps |
| Mycoplasma | Para influenza virus |
| Parvovirus | Pasteurella |
| Pertussis | Pestivirus |
| Plague | Pneumococcal |
| Polio (IPV) | Polio (OPV) |
| Pseudorabies | Rabies |
| Respiratory syncitial virus | Rotavirus |
| Rubella | Salmonella |
| Tetanus | Typhoid |
| Varicella | Yellow Fever |

Pharmaceuticals that may be applied in pharmaceutically active compositions according to the present invention may be further exemplified by low-molecular-weight drugs such as water-soluble anticancer agents, antibiotics, anti-inflammatory drugs, alkylatng agents, and immunosuppressants. Examples of these drugs include adriamycin, bleomycins, mitomycins, fluorouracil, peplomycin sulfate, daunorubicin hydrochloride, hydroxyurea, neocarzinostatin, sizofiran, estramustine phosphate sodium, carboplatin, beta-lactams, tetracyclines, aminoglycosides, and phosphomycin.

The pharmaceutically active composition of the present invention may contain two or more drugs depending on the disease and method of application.

For example, in veterinary applications for control of parasitic infections, a combination of ivermectin and praziquantel or a combination of zeranol and trembolone may be used.

Water-insoluble pharmaceutically active components which may be utilised in the sustained release delivery apparatus according to the present invention include lypophilic pharmaceuticals.

A lipophilic pharmaceutical may be any lipophilic substance so long as it is, as a form of a preparation, in a solid state at the body temperature of an animal or a human being to which the preparation is to be administered. “Lipophilic” as herein used means that the solubility of a substance in water is low, which specifically includes the following natures, as described in Pharmacopoeia of Japan 13th Edition (1996): practically insoluble (the amount of more than or equal to 10000 ml of solvent is required to dissolve 1 g or 1 ml of a solute), very hard to dissolve (the amount of more than or equal to 1000 ml and less than 10000 ml of solvent is required to dissolve 1 g or 1 ml of a solute), or hard to dissolve (the amount of more than or equal to 100 ml and less than 1000 ml of solvent is required to dissolve 1 g or 1 ml of a solute).

Specific examples of the lipophilic pharmaceutical include, but are not limited to, one or more selected from the group consisting of anti-parasitic agents (e.g. avermectin, ivermectin, spiramycin), antimicrobials (eg. ceftiofur; amoxicillin, erythromycin, oxytetracycline, and lincomycin), anti-inflammatory agents (eg. dexamethasone and phenylbutasone), hormones (eg. levothyroxine), adrenocorticosteroids (eg. dexamethasone palmitate, triamcinolone acetonide, and halopredone acetate), non-steroidal anti-inflammatory agents (eg. indometacin and aspirin), therapeutic agents for arterial occlusion (eg. prostaglandin E1), anticancer drugs (eg. actinomycin and daunomycin), therapeutic agents for diabetes (eg. acetohexamide), and therapeutic agents for osteopathy (eg. estradiol).

Depending on a disease or a method for application, multiple lipophilic drugs may be contained. In addition to the lipophilic drug having a direct therapeutic effect, the drug may be a substance with a biological activity, and such a substance as promotes or induces a biological activity, which includes an adjuvant for a vaccine, for example saponin. In such a case, incorporation of a vaccine into an implant results in a sustained release preparation of a vaccine with an adjuvant.

As stated above, the pharmaceutically active composition according to the present invention further includes a carrier for the pharmaceutically active component.

The pharmaceutical carrier may be selected to permit release of the pharmaceutically active component over an extended period of time from the composition.

The carrier may include a water-soluble substance.

A water-soluble substance is a substance which plays a role of controlling infiltration of water into the inside of the drug dispersion. There is no restriction in terms of the water-soluble substance so long as it is in a solid state (as a form of a preparation) at the body temperature of an animal or human being to which it is to be administered, and a physiologically acceptable, water-soluble substance.

One water-soluble substance, or a combination of two or more water-soluble substances may be used. The water-soluble substance specifically may be selected from one or more of the group consisting of synthetic polymers (eg. polyethylene glycol, polyethylene polypropylene glycol), sugars (eg. sucrose, mannitol, glucose, sodium chondroitin sulfate), polysaccharides (e.g. dextran), amino acids (eg. glycine and alanine), mineral salts (eg. sodium chloride), organic salts (eg. sodium citrate) and proteins (eg. gelatin and collagen and mixtures thereof).

In addition, when the water-soluble substance is an amphipathic substance, which dissolves in both an organic solvent and water, it has an effect of controlling the release of, for example, a lipophilic drug by altering the solubility thereof. An amphipathic substance includes, but not limited to, one or more selected from the group consisting of polyethylene glycol or a derivative thereof, polyoxyethylene polyoxypropylene glycol or a derivative thereof, fatty acid ester and sodium alkylsulfate of sugars, and more specifically, polyethylene glycol, polyoxy stearate 40, polyoxyethylene[196]polyoxypropylene [67]glycol, polyoxyethylene[105] polyoxypropylene[5]glycol, polyoxyethylene[160] polyoxypropylene[30]glycol, sucrose esters of fatty acids, sodium lauryl sulfate, sodium oleate, sodium chloride, sodium desoxycholic acid (or sodium deoxycholic acid (DCA)) of which mean molecular weights are more than 1500.

Polyoxyethylene polyoxypropyleneglycol, sucrose, sodium chloride or DCA or a mixture of two or more thereof are preferred.

In addition, the water-soluble substance may include a substance which is water-soluble and has any activity in vivo such as low molecular weight drugs, peptides, proteins, glycoproteins, polysaccharides, or an antigenic substance used as vaccines, i.e. water-soluble drugs.

The pharmaceutical carrier may constitute from approximately 1% to 30% by weight, preferably approximately 10% to 20% by weight, based on the total weight of the pharmaceutically active composition.

Each sustained release implant or mini-pellet may include additional carrier or excipients, lubricants, fillers, plasticisers, binding agent, colourants and stabilising agents.

Suitable fillers may be selected from the group consisting of talc, titanium dioxide, starch, kaolin, cellulose (microcrystalline or powdered) and mixtures thereof.

Suitable binding agents include polyvinyl pyrrolidine, hydroxypropyl cellulose and hydroxypropyl methyl cellulose and mixtures thereof.

The sustained release implant according to the present invention may have a rod-like shape, for example it is selected from circular cylinders, prisms, and elliptical cylinders. When the device is administered using an injector-type instrument, a circular cylindrical device is preferred since the injector body and the injection needle typically have a circular cylindrical shape.

The sustained release implant according to the present invention may be manufactured according to copending Australian provisional patent application PR7614 entitled “Preparation of sustained release pharmaceutical composition”, to Applicants, the entire disclosure of which is incorporated herein by reference.

The inner layer of the pharmaceutical formulation of the present invention, viewed in right section, may contain two or more layers containing different water-soluble pharmaceuticals. These layers may take the form of concentric circles with a single center of gravity or may appear as a plural number of inner layers whose respective centers of gravity lie at different points in the cross section. When the pharmaceutical formulation contains more than one inner layer there may be one or more pharmaceuticals present in the inner layers. For example, the pharmaceuticals may be present such that each layer contains a different pharmaceutical or there is more than one pharmaceutical in one or all of the inner layers.

The size of the pharmaceutical formulation of the present invention may, e.g. in the case of subcutaneous administration, be relatively small, e.g. ¼ to 1/10 normal size. For example using an injector-type instrument, the configuration may be circular cylindrical, and the cross-sectional diameter in the case is preferably 0.2 to 15 mm, the axial length being preferably approximately 0.2 to 7.5 mm, preferably approximately 0.5 to 5 mm, more preferably approximately 1 to 4 mm.

Sustained release implants according to the present invention may preferably have a double-layer structure, in order to achieve long-term zero-order release. The double layer structure may include

a pharmaceutical active-containing inner layer; and

a water impermeable outer layer.

The water impermeable outer layer may be formed of a silicone material. More preferably water-impermeable outer layers may be formed from a liquid coating composition including a liquid siloxane component.

Applicants have surprisingly found that the sustained release mini-implants having a double layer structure exhibit an unexpected release profile. Contrary to expectations, the maximum serum levels vary with the length of implant, not merely the time period over which sustained release is maintained (see Table 9). Whilst we do not wish to be restricted by theory, it is postulated that, particularly for small molecules, release is occurring not only from the open ends of the covered rod implant but also through the water-impermeable outer layer.

Such a release mechanism provides significant freedom in designing both the rate and time of release by simply varying implant length. Thus, for example, implants of varying sizes may be included to deliver a variety of desired treatment regimes.

Where a double-layer structure is used, the pharmaceutical-containing inner layer and the water-impermeable outer layer may be fabricated separately or simultaneously. A circular cylindrical sustained release apparatus with a single centre of gravity in the device cross section may be fabricated, for example, by the following methods:

(1) initial fabrication of a rod-shaped inner layer followed by coating the rod with a liquid containing dissolved outer layer material and drying;

(2) insertion of a separately fabricated inner layer into a tube fabricated from outer layer material; or

(3) simultaneous extrusion and molding of the inner and outer layers using a nozzle.

However, the fabrication method is not limited to these examples. When a water-impermeable outer layer cannot be obtained in a single operation, it will then be necessary, for example, to repeat the outer layer fabrication process until water permeation can be prevented. In any case, the resulting composition is subsequently cut into suitable lengths. Successive cutting yields a sustained release apparatus according to the present invention having both ends open.

Desirably the rod-like implant includes an outer coating layer. The thickness of the outer layer should be selected as a function of the material properties and the desired release rate. The outer layer thickness is not critical as long as the specified functions of the outer layer are fulfilled. The outer layer thickness is preferably approximately 0.05 mm to 3 mm, more preferably 0.05 mm to 0.25 mm, and most preferably 0.05 mm to 0.1 mm.

A pharmaceutical formulation with an open end at one terminal only may be fabricated by dipping one terminal of the pharmaceutical formulation into a solution which dissolves the outer-layer material and drying it, or by covering one terminal end of the pharmaceutical formulation with a cap made from the outer-layer material. In addition, the fabrication may comprise insertion of the inner layer into an outer-layer casing with a closed-end at one terminal, which are separately produced, and also formation of the inner layer in said casing.

In a further aspect of the present invention there is provided a method for the therapeutic or prophylactic treatment of an indication in an animal (including a human) requiring such treatment, which method includes administering to the animal a sustained release delivery apparatus including a plurality of sustained release mini-implants or pellets;

each mini-implant including

a sustained release support material; and

a pharmaceutically active composition carried in or on the sustained release support material;

the pharmaceutically active composition including

at least one pharmaceutically active component; and

a carrier therefor;

each implant being of insufficient size individually to provide a predetermined desired threshold blood level of pharmaceutical active for treatment of a selected indication.

As stated above, it has been found that the pharmaceutical payload may be increased by the sustained release delivery apparatus according to the present invention when compared to the prior art. For example, diseases which were heretofore untreatable may now be treated over an extended period of time utilising the apparatus of the present invention.

For example, in animals suffering from parasitic infections such as ticks, the animals may be treated utilising the sustained release delivery apparatus including an anti-parasitic drug such as ivermectin. Heretofore, it was not possible to achieve a required blood concentration threshold to permit treatment of such a parasitic disease utilising a sustained release approach as the required blood concentration threshold could not be achieved utilising such a mechanism.

Preferably, the mini-implants or pellets are provided in at least two different sizes.

More preferably the mini-implants or pellets are provided

in a first size which provides a blood level of pharmaceutical active of approximately 1.25 to 3 times the desired threshold blood level for a first relatively short time period; and

in a second size which provides a blood level of pharmaceutical active at or near the desired threshold blood level for a second longer time period.

In a further preferred embodiment each mini-implant includes

an inner pharmaceutical active-containing inner layer; and

a water-impermeable outer layer.

The method of administration may include subcutaneous or intramuscular injection, intradermal injection, intraperitoneal injection, intraocular or in the ear, intranasal insertion or indwelling, intravaginal or intradwelling, intrarectal insertion or indwelling, for example as a suppository or utilising oral administration.

The animals to be treated may be selected from the group consisting of sheep, cattle, goats, horses, camels, pigs, dogs, cats, ferrets, rabbits, marsupials, buffalos, yacks, primates, humans, birds including chickens, geese and turkeys, rodents including rats and mice, fish, reptiles and the like.

The method according to the present invention is particularly applicable to larger animals, e.g. cattle, sheep, pigs, dogs and humans where high dosage levels are required to achieve the prerequisite threshold pharmaceutical active blood levels for successful treatment of selected disease indications.

The present invention will now be more fully described with reference to the accompanying examples. It should be understood, however, that the description following is illustrative only and should not be taken in anyway as a restriction on the generality of the invention described above.

### EXAMPLE 1

A mixture of ivermectin and carrier material in proportions specified in Table 1 below was produced. The obtained solid was milled and passed through a sieve (212 μm). A portion of a powder thus obtained and Silastic™ Medical Grade ErR Elastomer Q7-4750 Component A and Silastic™ Medical Grade ETR Elastomer Q7-4750 component B were mixed to give a drug dispersion component. Silastic™ Medical Grade ETR Elastomer Q7-4750 Component A and Silastic™ Medical Grade ETR Elastomer 07-4750 Component B were mixed to give a coating layer component. Thus obtained drug dispersion component and coating layer component were molded by extruding from a double extruder which enables them to be molded by extruding so that the drug dispersion is concentrically coated with the coating layer, and was allowed to stand at room temperature to cure, which was cut to obtain the cylindrical preparation 1 (the length of the preparation is 500 mm, the diameter of the preparation is 3 or 4 mm).

TABLE 1

| | | | | Composition/ | | | | Ivermectin | Total |
|---|---|---|---|---|---|---|---|---|---|
| | | Diameter | Powder | Powder (%) | | | | (IVM) Content | length |
| Sample No | Type | (mm) | (%) | IVM | PEPPG | DCA | SUC | (mg/mm) | (mm) |
| 1 | CR | 3 | 50 | 85 | 15 | — | — | 2.45 | 500 |
| 2 | CR | 3 | 50 | 70 | 30 | — | — | 1.99 | 500 |
| 3 | CR | 4 | 50 | 85 | 15 | — | — | 4.26 | 500 |
| 4 | CR | 3 | 40 | 80 | — | 13 | 7 | 1.89 | 500 |
| 5 | CR | 3 | 50 | 80 | — | 13 | 7 | 2.43 | 500 |
| 6 | CR | 3 | 50 | 75 | — | 25 | — | 2.13 | 500 |
| 7 | CR | 3 | 50 | 75 | — | — | 25 | 2.23 | 500 |
| 8 | M | 3 | 50 | 75 | — | 25 | — | 3.15 | 500 |
| 9 | CR | 3 | 30 | 50 | — | 33 | 17 | 1.06 | 500 |

Abbreviations
CR = covered rod
M = matrix
IVM = Ivermectin
PEPPG = polyoxyethylene polyoxypropylene glycol
DCA = sodium deoxycholic acid
SUC = sucrose

The cylindrical preparation 1 is then cut into various lengths as shown in Tables 2 to 5A to provide the sustained release mini-pellets according to the present invention.

Examination 1

Preparation 1 was subcutaneously administered to various animals including dogs, sheep and cattle, whole blood was collected from the animal via the jugular vein and in the case of rats under anaesthesia with ether at the day of determination, and then, the concentration of ivermectin in the plasma was determined by high performance liquid chromatography.

TABLE 2A
CATTLE

| | | | | | | | | Treatment A - 8 cm | | | | Treatment B - 4 cm | | | |
|---|---|---|---|---|---|---|---|---|---|---|---|---|---|---|---|
| | | | | | | | | 4 cm | 1 cm | 0.4 cm | 0.2 cm | 4 cm | | 0.4 cm | 0.2 cm |
| | | Powder | Composition | | | | 4 cm | 2 × 4 | 2 × 4 × | 2 × 10 × | 2 × 20 × | 1 × 4 | 1 cm | 1 × 10 × | 1 × 20 × |
| Implant | Type | % | IVM | PEPPG | DCA | SUC | dose | cm | 1 cm | 0.4 cm | 0.2 cm | cm | 4 × 1 cm | 0.4 cm | 0.2 cm |
| JN-96Ab | CR | 50 | 85 | 15 | — | — | 98 | 1 | 2 | 3 | 4 | 5 | 6 | 7 | 8 |
| JN-96Bb | CR | 50 | 70 | 30 | — | — | 80 | 9 | 10 | 11 | 12 | 13 | 14 | 15 | 16 |
| JN-97Db* | CR | 50 | 85 | 15 | — | — | 170 | 17 | 18 | 19 | 20 | 21 | 22 | 23 | 24 |
| JN-96Ea | CR | 40 | 80 | — | 13 | 7 | 76 | 25 | 26 | 27 | 28 | 29 | 30 | 31 | 32 |
| JN-96Eb | CR | 50 | 80 | — | 13 | 7 | 97 | 33 | 34 | 35 | 36 | 37 | 38 | 39 | 40 |
| JN-96Hb | CR | 50 | 75 | — | 25 | — | 85 | 41 | 42 | 43 | 44 | 45 | 46 | 47 | 48 |
| JN-96Ib | CR | 50 | 75 | — | — | 25 | 89 | 49 | 50 | 51 | 52 | 53 | 54 | 55 | 56 |
| JN-96Kb | M | 50 | 75 | — | 25 | — | 126 | 57 | 58 | 59 | 60 | 61 | 62 | 63 | 64 |
| JN-080-M | CR | 30 | 50 | — | 33 | 17 | 42 | 65 | 66 | 67 | 68 | 69 | 70 | 71 | 72 |
| Placebo | | | | | | | 0 | 73 | 74 | 75 | 76 | 77 | 78 | 79 | 80 |

TABLE 2B
CATTLE

| Treat- | | | | | | | | | | |
|---|---|---|---|---|---|---|---|---|---|---|
| ment | | Ivermectin (ng/ml) | | | | | | | | |
| numb- | | Weeks | | | | | | | | |
| er | Implant | 1 | 2 | 4 | 6 | 8 | 10 | 12 | 14 | 16 |
| 1 | JN-096- | 7.3 | 5.4 | 3.3 | 3.6 | | | 0.61 | | |
| 2 | Ab | 5.4 | 2.1 | | 2.9 | | | 0.68 | | |
| 3 | | 6.9 | 6.9 | 5.5 | 6.2 | | | 1.1 | | |
| 4 | | 4.8 | 7.0 | 5.7 | 6.0 | | | 1.2 | | |
| 5 | | 1.5 | 1.9 | 2.1 | 1.8 | | | 0.46 | | |
| 6 | | 2.6 | 2.9 | 4.0 | 3.5 | | | 0.51 | | |
| 7 | | 3.8 | 4.2 | 2.8 | 3.5 | | | 0.5 | | |
| 8 | | 4.4 | 5.2 | 5.8 | 3.8 | | | 0.73 | | |
| 9 | JN-096- | 2.9 | 2.9 | ND | ND | | | ND | | |
| 10 | Bb | 3.1 | 5.3 | 4.3 | 1.6 | | | 1.1 | | |
| 11 | | 8.4 | 11.0 | 8.3 | 3.8 | | | 1.5 | | |
| 12 | | 13 | 13.0 | 19.0 | 7.6 | | | 1.3 | | |
| 13 | | 4.6 | 4.1 | 2.6 | 1.3 | | | 0.54 | | |
| 14 | | 5.9 | 5.1 | 3.3 | 1.5 | | | 0.71 | | |
| 15 | | 8.6 | 8.6 | 6.4 | 2.6 | | | 0.66 | | |
| 16 | | 3.1 | 6.1 | 4.6 | 2.1 | | | 0.79 | | |
| 17 | JN-096- | 8.0 | 8.2 | 4.5 | 3.7 | | | 1.2 | | |
| 18 | Db | 12.0 | 10.0 | 6.3 | 5.6 | | | 1.2 | | |
| 19 | | 13.0 | 19.0 | 24 | 17 | | | 2.6 | | |
| 20 | | 13.9 | 18.0 | 10 | 8.8 | | | 3.0 | | |
| 21 | | 4.5 | 3.8 | 2.5 | 3.1 | | | 0.47 | | |
| 22 | | 4.3 | 4.3 | 2.6 | 2.5 | | | 0.57 | | |
| 23 | | 5.4 | 9.0 | 5.0 | 4.6 | | | 0.8 | | |
| 24 | | 15.0 | 15.0 | 10.0 | 8.0 | | | 0.65 | | |
| 25 | JN-096- | 5.0 | 4.8 | 1.7 | 2.0 | | | 1.2 | | |
| 26 | Ea | 7.6 | 5.2 | 3.8 | 2.6 | | | 1.0 | | |
| 27 | | 5.5 | 7.1 | 4.0 | 4.1 | | | 0.82 | | |
| 28 | | 11.0 | 13.0 | 7.4 | 5.9 | | | 1.4 | | |
| 29 | | 3.2 | 2.6 | 2.1 | 2.1 | | | 1.9 | | |
| 30 | | 2.5 | 2.1 | 1.7 | 1.4 | | | 0.5 | | |
| 31 | | 4.4 | 4.9 | 2.8 | 3.1 | | | 0.51 | | |
| 32 | | 4.5 | 5.2 | 3.1 | 3.0 | | | 0.44 | | |
| 33 | JN-096- | 5.4 | 4.6 | 3.3 | 3.1 | | | 0.92 | | |
| 34 | Eb | 9.1 | 10 | 5.3 | 5.2 | | | 1.3 | | |
| 35 | | 4.4 | 4.3 | 8.3 | 6.5 | | | 0.38 | | |
| 36 | | 4.4 | 8.4 | 6.5 | 7.4 | | | 2.1 | | |
| 37 | | | 2.4 | 2.0 | 2.3 | | | 0.41 | | |
| 38 | | 2.3 | 2.5 | 1.3 | 1.7 | | | 0.3 | | |
| 39 | | 4.5 | 5.6 | 2.4 | 2.3 | | | 0.39 | | |
| 40 | | 3.2 | 5.6 | 4.9 | 3.8 | | | 0.58 | | |
| 41 | JN-096- | 3.3 | 4.3 | 5.5 | 5.0 | | | 0.87 | | |
| 42 | Hb | 7.8 | 7.8 | 6.2 | 5.3 | | | 1.7 | | |
| 43 | | 4.3 | 4.7 | 3.9 | 2.6 | | | 0.7 | | |
| 44 | | 5.0 | 11.0 | 12.0 | 6.8 | | | 1.4 | | |
| 45 | | 1.8 | 2.4 | 1.5 | 1.5 | | | 0.46 | | |
| 46 | | 3.7 | 4.3 | 2.8 | 2.1 | | | 0.73 | | |
| 47 | | 3.5 | 6.4 | 4.9 | 4.2 | | | 0.74 | | |
| 48 | | 4.4 | 4.3 | 3.9 | 3.1 | | | 0.74 | | |
| 49 | JN-096- | 3.3 | 2.9 | 1.8 | 0.88 | | | 0.49 | | |
| 50 | Ib | | 2.3 | 2.1 | 1.6 | | | 0.72 | | |
| 51 | | 3.3 | 5.2 | 4.6 | 4.0 | | | 0.66 | | |
| 52 | | 4.3 | 3.7 | 0.31 | ND | | | ND | | |
| 53 | | 1.5 | 1.5 | 1.2 | 0.90 | | | 0.25 | | |
| 54 | | 2.3 | 2.6 | 1.7 | 1.1 | | | 0.36 | | |
| 55 | | 2.3 | 2.2 | 2.0 | 1.5 | | | 3.6 | | |
| 56 | | 3.8 | 3.5 | 3.6 | 2.4 | | | 0.63 | | |
| 57 | JN-096- | 2.3 | 1.3 | ND | ND | | | ND | | |
| 58 | Kb | ND | 2.1 | 3.2 | 1.4 | | | ND | | |
| 59 | | 3.8 | 4.4 | 1.5 | 0.49 | | | ND | | |
| 60 | | 9.0 | 5.5 | 0.52 | ND | | | ND | | |
| 61 | | 17.0 | 13.0 | 6.2 | 3.4 | | | 1.0 | | |
| 62 | | 2.2 | 3.0 | 3.0 | 2.3 | | | 0.63 | | |
| 63 | | 3.2 | 6.9 | 4.7 | 2.1 | | | 0.66 | | |
| 64 | | 2.1 | 3.1 | 4.4 | 2.4 | | | 1.2 | | |
| 65 | JN-080- | 3.3 | 2.9 | 2.8 | 2.1 | | | 0.76 | | |
| 66 | M | 4.6 | 4.8 | 6.1 | 3.6 | | | 0.75 | | |
| 67 | | 8.8 | 9.0 | 6.4 | 5.4 | | | 1.3 | | |
| 68 | | 5.5 | 5.1 | 8.4 | 5.0 | | | 0.84 | | |
| 69 | | 2.5 | 2.0 | 1.7 | 1.5 | | | 0.38 | | |
| 70 | | 2.7 | 2.3 | 3.5 | 1.6 | | | 0.75 | | |
| 71 | | 2.0 | 2.9 | 1.8 | 2.5 | | | 0.29 | | |
| 72 | | 2.9 | 1.7 | 2.6 | 2.4 | | | 0.58 | | |
| 73 | Control | ND | ND | ND | ND | | | ND | | |
| 74 | | ND | ND | ND | ND | | | ND | | |
| 75 | | ND | ND | ND | ND | | | — | | |
| 76 | | 5.3 | ND | ND | ND | | | ND | | |
| 77 | | ND | ND | ND | | | | ND | | |
| 78 | | ND | ND | ND | | | | ND | | |
| 79 | | ND | ND | ND | | | | ND | | |
| 80 | | ND | ND | ND | | | | ND | | |

TABLE 3A
SHEEP

| | | | Composition | | | | | Treatment - 4 cm | | |
|---|---|---|---|---|---|---|---|---|---|---|
| Implant | Type | Powder % | Diameter | IVM | DCA | SUC | 4 cm dose | 4 × 1 cm | 4 × 2 × 0.5 cm | 4 × 4 × 0.25 cm |
| JN-095A | CR | 30 | 2 mm | 100 | — | — | 37.6 | 1 | 2 | 3 |
| JN-095B | CR | 30 | 2 mm | 75 | — | 25 | 28.4 | 4 | 5 | 6 |
| JN-095G | CR | 30 | 2 mm | 50 | 50 | — | 18.8 | 7 | 8 | 9 |
| JN-095F | CR | 30 | 2 mm | 25 | 25 | — | 28.4 | 10 | 11 | 12 |
| JN-080-M | CR | 30 | 3 mm | 50 | 33 | 17 | 42.0 | 13 | 14 | 15 |

TABLE 3B
SHEEP

| | Ivermectin (ng/ml) | | | | | |
|---|---|---|---|---|---|---|
| | Week | | | | | |
| | 0 | Week 1 | Week 2 | Week 3 | Week 4 | Week 6 |
| JN-095A 1 cm | 0 | ND | 0.61 | 0.55 | 0.38 | 0.21 |
| JN-095A 0.5 cm | 0 | 1 | 1 | 0.78 | 0.57 | 0.38 |
| JN-095A 0.25 cm | 0 | ND | 0.78 | 0.65 | 0.56 | 0.4 |
| JN-095B 1 cm | 0 | ND | 0.76 | 0.54 | 0.36 | 0.25 |
| JN-095B 0.5 cm | 0 | 1.5 | 1.3 | 1.1 | 0.79 | 0.40 |
| JN-095B 0.25 cm | 0 | 2.1 | 1.5 | 1.1 | 0.69 | 0.51 |
| JN-095G 1 cm | 0 | ND | 0.75 | 0.53 | 0.48 | 0.34 |
| JN-095G 0.5 cm | 0 | 1.8 | 1.6 | 1.5 | 1.1 | 0.75 |
| JN-095G 0.25 cm | 0 | 2.9 | 2.6 | 1.7 | 1.1 | 0.62 |
| JN-095F 1 cm | 0 | 1.5 | 1.4 | 1.2 | 0.94 | 0.64 |
| JN-095F 0.5 cm | 0 | ND | 0.71 | 0.67 | 0.39 | 0.32 |
| JN-095F 0.25 cm | 0 | 1.6 | 1.1 | 1 | 0.66 | 0.54 |
| JN-080M 1 cm | 0 | ND | ND | ND | ND | ND |
| JN-080M 0.5 cm | 0 | 2.3 | 2.5 | 1.9 | 0.93 | 0.28 |
| JN-080M 0.25 cm | 0 | 4.5 | 3.7 | 2.6 | 1.4 | 0.81 |

TABLE 4A
DOG

| | | Pow- | | | | | Treatment A - 2.4 cm | | | | Treatment B - 1.2 cm | | | |
|---|---|---|---|---|---|---|---|---|---|---|---|---|---|---|
| | | der | Composition | | | 1.2 cm | 1.2 cm + | 1.2 cm + | 1.2 cm + | 1.2 cm + | | 2 × 0.6 | 3 × 0.4 | 6 × 0.2 |
| Implant | Type | % | IVM | DCA | SUC | dose | 1.2 cm | 2 × 0.6 cm | 3 × 0.4 cm | 6 × 0.2 cm | 1.2 cm | cm | cm | cm |
| JN-090B | CR | 30 | 90 | — | 10 | 5.16 | 1 | 2 | 3 | 4 | 5 | 6 | 7 | 8 |
| JN-090E | CR | 30 | 90 | 6.5 | 3.5 | 5.28 | 9 | 10 | 11 | 12 | 13 | 14 | 15 | 16 |
| JN-090D | CR | 30 | 80 | — | 20 | 4.56 | 17 | 18 | 19 | 20 | 21 | 22 | 23 | 24 |
| JN-090F | CR | 30 | 80 | 13 | 7 | 4.68 | 25 | 26 | 27 | 28 | 29 | 30 | 31 | 32 |

TABLE 4B
DOG

| | Total | Length | | Ivermectin (ng/ml) | | | | | | | |
|---|---|---|---|---|---|---|---|---|---|---|---|
| Implant | Length | Combinations | Treat No | Week 1 | Week 2 | Week 3 | Week 4 | Week 6 | Week 8 | Week 10 | Week 12 |
| JN-090-B | 2.4 | 1.2 × 2 | 1 | 2.9 | 3.9 | 2.6 | 2.0 | | 1.2 | | |
| JN-090-B | 2.4 | 1.2 × 1, 0.6 × 2 | 2 | 4.5 | 4.8 | 3.3 | 2.6 | | 1.5 | | |
| JN-090-B | 2.4 | 1.2 × 1, 0.4 × 3 | 3 | 4.9 | 4.8 | 3.1 | 1.8 | | 0.38 | | |
| JN-090-B | 2.4 | 1.2 × 1, 0.2 × 6 | 4 | 8.0 | 7.1 | 4.1 | 3.1 | | 1.4 | | |
| JN-090-B | 1.2 | 1.2 × 1 | 5 | 2.2 | 2.1 | 1.1 | 1.0 | | 0.34 | | |
| JN-090-B | 1.2 | 0.6 × 2 | 6 | 2.0 | 1.8 | 1.1 | 0.84 | | 0.26 | | |
| JN-090-B | 1.2 | 0.4 × 3 | 7 | 4.0 | 3.0 | 1.5 | 0.88 | | 0.4 | | |
| JN-090-B | 1.2 | 0.2 × 6 | 8 | 2.1 | 1.6 | 1.1 | 0.45 | | 0.3 | | |
| JN-090-E | 2.4 | 1.2 × 2 | 9 | 6.1 | 7.3 | 6.0 | 4.7 | | 3.3 | | |
| JN-090-E | 2.4 | 1.2 × 1, 0.6 × 2 | 10 | 3.9 | 4.4 | 5.4 | 4.6 | | 5.3 | | |
| JN-090-E | 2.4 | 1.2 × 1, 0.4 × 3 | 11 | 5.7 | 6.2 | 5.1 | 4.2 | | 2.8 | | |
| JN-090-E | 2.4 | 1.2 × 1, 0.2 × 6 | 12 | 8.7 | 7.4 | 5.0 | 4.6 | | 2.4 | | |
| JN-090-E | 1.2 | 1.2 × 1 | 13 | 1.7 | 2.2 | 1.8 | 1.3 | | 1.7 | | |
| JN-090-E | 1.2 | 0.6 × 2 | 14 | 2.6 | 2.9 | 1.7 | 2.0 | | 2.2 | | |
| JN-090-E | 1.2 | 0.4 × 3 | 15 | 1.9 | 1.8 | 1.2 | 1.2 | | 0.91 | | |
| JN-090-E | 1.2 | 0.2 × 6 | 16 | 3.9 | 2.7 | 2.4 | 2.3 | | 1.6 | | |
| JN-090-D | 2.4 | 1.2 × 2 | 17 | 4.3 | 5.9 | 3.6 | 1.7 | | 1.0 | | |
| JN-090-D | 2.4 | 1.2 × 1, 0.6 × 2 | 18 | 5.6 | 5.4 | 5.4 | 3.7 | | 1.7 | | |
| JN-090-D | 2.4 | 1.2 × 1, 0.4 × 3 | 19 | 6.2 | 5.8 | 4.5 | 3.0 | | 1.0 | | |
| JN-090-D | 2.4 | 1.2 × 1, 0.2 × 6 | 20 | 11 | 7.1 | 4.3 | 3.0 | | 0.88 | | |
| JN-090-D | 1.2 | 1.2 × 1 | 21 | NT | 2.1 | 1.7 | 1.5 | | 0.46 | | |
| JN-090-D | 1.2 | 0.6 × 2 | 22 | 2.8 | 2.3 | 1.6 | 1.0 | | 0.37 | | |
| JN-090-D | 1.2 | 0.4 × 3 | 23 | 2.9 | 2.2 | 1.4 | 1.3 | | 0.85 | | |
| JN-090-D | 1.2 | 0.2 × 6 | 24 | 4.0 | 4.0 | 2.5 | 1.8 | | 0.44 | | |
| JN-090-F | 2.4 | 1.2 × 2 | 25 | 3.2 | 4.4 | 3.8 | 4.0 | | 2.9 | | |
| JN-090-F | 2.4 | 1.2 × 1, 0.6 × 2 | 26 | NS | 6.3 | 5.6 | 5.6 | | 4.9 | | |
| JN-090-F | 2.4 | 1.2 × 1, 0.4 × 3 | 27 | 6.1 | 8.2 | 7.0 | 4.9 | | 3.0 | | |
| JN-090-F | 2.4 | 1.2 × 1, 0.2 × 6 | 28 | 9.9 | 11.0 | NS | 5.6 | | Died | | |
| JN-090-F | 1.2 | 1.2 × 1 | 29 | NS | 3.5 | 2.6 | 2.3 | | 0.94 | | |
| JN-090-F | 1.2 | 0.6 × 2 | 30 | 1.5 | 2.1 | 2.0 | 1.2 | | 1.1 | | |
| JN-090-F | 1.2 | 0.4 × 3 | 31 | 3.1 | 3.8 | 3.4 | 3.0 | | 1.4 | | |
| JN-090-F | 1.2 | 0.2 × 6 | 32 | 5.8 | 6.2 | 4.4 | 3.9 | | 2.8 | | |

TABLE 5A
DOG

| Data at Week 1 - Dog | | | | | | | | | | | | | | |
|---|---|---|---|---|---|---|---|---|---|---|---|---|---|---|
| (serum concentration ng/ml) | | | | | | | Treatment A - 2.4 cm | | | | Treatment B - 1.2 cm | | | |
| | | Powder | Composition | | | 1.2 cm | 1.2 cm + | 1.2 cm + | 1.2 cm + | 1.2 cm + | | 2 × 0.6 | 3 × 0.4 | 6 × 0.2 |
| Implant | Type | % | IVM | DCA | SUC | dose | 1.2 cm | 2 × 0.6 cm | 3 × 0.4 cm | 6 × 0.2 cm | 1.2 cm | cm | cm | cm |
| 1 | CR | 30 | 90 | — | 10 | 5.16 | 2.9 | 4.5 | 4.9 | 8.0 | 2.2 | 2.0 | 4.0 | 2.1 |
| 2 | CR | 30 | 90 | 6.5 | 3.5 | 5.28 | 6.1 | 3.9 | 5.7 | 8.7 | 1.7 | 2.6 | 1.9 | 3.9 |
| 3 | CR | 30 | 80 | — | 20 | 4.56 | 4.3 | 5.6 | 6.2 | 11 | NT | 2.8 | 2.9 | 4.0 |
| 4 | CR | 30 | 80 | 13 | 7 | 4.68 | 3.2 | NS | 27 | 28 | 29 | 30 | 31 | 32 |

Abbreviations
CR = covered rod
M = matrix
IVM = Ivermectin
PEPPG = polyoxyethylene polyoxypropylene glycol
DCA = sodium deoxycholic acid
SUC = sucrose
NT = not tested
NS = no sample

### EXAMPLE 2

Rat Experiment

Experimental Protocol

Rats (Sprague Dawley) were allocated to 7 groups and implanted with implants of different lengths that corresponded to a final dose of ivermectin between 2 and 20 mg/kg (1 to 10 mg per rat). A single rat from each group was sacrificed at various time points, and a serum sample collected.

Starting ivermectin content of implants of varying lengths are set out in Table 6 with formulation details in Table 7.

TABLE 6
Starting ivermectin (mg) of implants cut to various lengths

| Implant length | JN-090-E |
|---|---|
| 0.2 cm | 0.88 |
| 0.4 cm | 1.76 |
| 0.6 cm | 2.64 |
| 0.8 cm | 3.52 |
| 1.0 cm | 4.40 |
| 1.5 cm | 6.60 |
| 2.0 cm | 8.80 |

TABLE 7
Implant formulation tested

| | | Composition/ | | | | |
|---|---|---|---|---|---|---|
| Implant | Type of | Powder % | | | | Ivermectin (IVM) |
| number | implant | IVM | DOC | SUC | Diameter | content (mg/mm) |
| JN-090-E | CR | 90 | 6.5 | 3.5 | 1.5 mm | 0.44 |

The results achieved are set out in Tables 8 and 9.

TABLE 8
Group weights and Ivermectin dosage (mg/kg)

| JN-090-E | Weight (g) | Dose (mg/kg) |
|---|---|---|
| 0.2 cm | 426 + 11 | 2.1 + 0.1 |
| 0.4 cm | 411 + 44 | 4.3 + 0.5 |
| 0.6 cm | 411 + 13 | 6.4 + 0.2 |
| 0.8 cm | 417 + 20 | 8.5 + 0.4 |
| 1.0 cm | 396 + 20 | 11.1 + 0.6 |
| 1.5 cm | 379 + 21 | 17.4 + 0.9 |
| 2.0 cm | 399 + 34 | 22.2 + 1.8 |

TABLE 9
Serum Ivermectin levels (μg/ml)

| | Maximum | Number of | Projected | Period (weeks) |
|---|---|---|---|---|
| | IVM | implants | serum | maximum |
| | serum level | to be | level of 2 cm | serum |
| JN-090-E | (μg/ml) | 2 cm | Implant (μg/ml) | level maintained |
| 0.2 cm | 3.2 | 10 | 32 | 1 |
| 0.4 cm | 6.7 | 5 | 33.5 | 1 |
| 0.6 cm | 5.8 | 3.3 | 19 | 2 |
| 0.8 cm | 7.1 | 2.5 | 17.8 | 3 |
| 1.0 cm | 8.4 | 2 | 16.8 | 4 |
| 1.5 cm | 8.6 | 1.3 | 11.2 | 22 |
| 2. cm | 11.0 | 1.0 | 11.0 | 24 |

Conclusions

1 The use of implants, the subject of the present invention (the division of a single larger implant into multiple small implants) results in a higher serum level of ivermectin over a shorter time frame (see Table 9).

2 The use of a larger implant (e.g. 1.5 or 2 cm) results in a higher serum level being maintained over a longer time frame, however the peak serum level is only “1/3” of that achieved if the same large implant is divided into multiple small implants (e.g. 0.2 cm or 0.4 cm).

3 The results of Table 9 clearly show

(a) that the use of implants the subject of the present invention results in more rapid release of IVM from the smaller implants

(b) that all the implants release ivermectin from the ends of the covered rods and from the sides of the covered rods (hence the higher serum levels achieved as the implants increase in length). This means that IVM can diffuse through the walls of the covered rod despite the presence of an aqueous impervious layer of silicon.

It will be understood that the invention disclosed and defined in this specification extends to all alterative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and may be used interchangeably and should not be taken as excluding the presence of other elements or features.

1-51. Cancelled.

52. A sustained release apparatus including a plurality of sustained release mini-implants or pellets;
each mini-implant including
a sustained release support material; and
a pharmaceutically active composition carried in or on the sustained release support material;
the pharmaceutically active composition including
at least one pharmaceutically active component; and
a carrier therefor;
each implant being of insufficient size and/or payload individually to provide a predetermined desired threshold blood level of pharmaceutical active for treatment of a selected indication;
the size(s) of the mini-implants or pellets providing zero order release of pharmaceutical active;
the sustained release apparatus providing, in use, zero order release of pharmaceutically active at, or above, the desired threshold level of pharmaceutical active for treatment of a selected indication.

53. A sustained release apparatus according to claim 52, wherein each mini-implant is of the uncovered or covered rod, or matrix type.

54. A sustained release apparatus according to claim 53, wherein each mini-implant includes
a pharmaceutical active-containing inner layer; and
a water-impermeable outer layer.

55. A sustained release apparatus according to claim 54, wherein each mini-implant takes the form of an extruded rod bearing a water-impermeable coating thereover, the water-impermeable coating being formed from a liquid coating composition including a liquid siloxane component.

56. A sustained release apparatus according to claim 52, wherein each mini-implant is approximately 0.1 to 0.5 times the length of a single rod shaped implant capable of providing the desired threshold blood level, depending on the pharmaceutical active selected.

57. A sustained release apparatus according to claim 56, wherein each mini-implant is approximately 0.20 to 0.5 times the length and/or diameter of a single rod shaped implant capable of providing the desired threshold blood level, depending on the pharmaceutical active selected.

58. A sustained release apparatus according to claim 57, wherein the sustained release mini-implant is of generally circular cylindrical configuration with a cross-sectional diameter of approximately 0.2 to 15 mm and an axial length of approximately 0.2 to 7.5 mm.

59. A sustained release apparatus according to claim 58, wherein the axial length of the mini-implant is approximately 0.5 to 5 mm.

60. A sustained release apparatus according to claim 52, wherein the mini-implants or pellets are provided in a first size which provides a blood level of pharmaceutical active of approximately 1.25 to 3 times the desired threshold blood level for a first relatively short time period; and
in a second size which provides a blood level of pharmaceutical active at or near the desired threshold blood level for a second longer time period.

61. A sustained release apparatus according to claim 60, wherein the first time period is of approximately 1 to 4 weeks and the second time period is of approximately 4 to 52 weeks.

62. A sustained release apparatus according to claim 52, wherein the pharmaceutically active composition includes at least one pharmaceutically active component selected from the group consisting of acetonemia preparations, anabolic agents, anaesthetics, analgesics, anti-acid agents, anti-arthritic agents, antibodies, anti-convulsivants, anti-fungals, anti-histamines, anti-infectives, anti-inflammatories, anti-microbials, anti-parasitic agents, anti-protozoals, anti-ulcer agents, antiviral pharmaceuticals, behaviour modification drugs, biologicals, blood and blood substitutes, bronchodilators and expectorants, cancer therapy and related pharmaceuticals, cardiovascular pharmaceuticals, central nervous system pharmaceuticals, coccidiostats and coccidiocidals, contraceptives, contrast agents, diabetes therapies, diuretics, fertility pharmaceuticals, growth hormones, growth promoters, hematinics, hemostatics, hormone replacement therapies, hormones and analogs, immunostimulants, minerals, muscle relaxants, natural products, nutraceuticals and nutritionals, obesity therapeutics, ophthalmic pharmaceuticals, osteoporosis drugs, pain therapeutics, peptides and polypeptides, respiratory pharmaceuticals, sedatives and tranquilizers, transplantation products, urinary acidifiers, vaccines and adjuvants and vitamins.

63. A sustained release apparatus according to claim 62 wherein the pharmaceutically active component includes an anti-parasitic agent which is a macrocyclic lactone, or insect growth regulator, or mixtures thereof.

64. A sustained release apparatus according to claim 63 wherein the macrocyclic lactone component includes ivermectin.

65. A sustained release apparatus according to claim 52, wherein the pharmaceutical carrier is selected to permit release of the pharmaceutically active component from the composition over an extended period of time.

66. A sustained release apparatus according to claim 65, wherein the pharmaceutical carrier includes a water-soluble substance which is in a solid state in the pharmaceutically active composition at the body temperature of an animal or human being to which it is to be administered.

67. A sustained release apparatus according to claim 66, wherein the pharmaceutical carrier is selected from one or more of the group consisting of synthetic polymers, sugars, amino acids, mineral salts, organic salts and proteins.

68. A sustained release apparatus according to claim 67, wherein the pharmaceutical carrier is a sugar or mineral salt or mixture thereof.

69. A sustained release apparatus according to claim 68, wherein when the pharmaceutically active composition includes a lipophilic pharmaceutical, the pharmaceutical carrier includes one or more amphipathic substances selected from the group consisting of one or more of polyoxy stearate 40, polyoxyethylene polyoxypropylene glycol, sucrose esters of fatty acids, sodium lauryl sulfate, sodium oleate, sodium chloride and sodium desoxycholic acid.

70. A sustained release apparatus according to claim 69, wherein the carrier includes polyoxyethylene polyoxypropylene glycol, sucrose, sodium chloride, or sodium desoxycholic acid or a mixture of two or more thereof.

71. A sustained release apparatus according to claim 65, wherein the pharmaceutical carrier constitutes from approximately 10 to 30% by weight, based on the total weight of the pharmaceutically active composition.

72. A sustained release apparatus according to claim 52, wherein the sustained release support material takes the form of a support matrix, tablet or rod formed from a biocompatible material selected from the group consisting of polyesters, polyamino acids, silicones, ethylene-vinyl acetate copolymers, poly(glycerol-sebacate) and polyvinyl alcohols.

73. A sustained release apparatus according to claim 72, wherein the sustained release support material includes a silicone material.

74. A sustained release apparatus according to claim 73, wherein the silicone material is formed from a methyl-vinyl siloxane polymer including a fumed silica as reinforcing filler.

75. A sustained release kit including a plurality of sustained release mini-implants or pellets packaged for delivery in a single treatment,
each mini-implant including
a pharmaceutically active composition including at least one pharmaceutically active component; and
a carrier therefor; and
a sustained release support material, the pharmaceutically active composition being carried in or on the sustained release support material;
each implant being of insufficient size and/or payload individually to provide a predetermined required threshold blood level of pharmaceutical active for treatment of a selected indication;
the size(s) of the mini-implants or pellets providing zero order release of the pharmaceutical active.

76. A sustained release kit according to claim 75, wherein the mini-implants or pellets are provided in a first size which provides a blood level of pharmaceutical active of approximately 1.25 to 3 times the desired threshold blood level for a first relatively short time period; and
in a second size which provides a blood level of pharmaceutical active at or near the desired threshold blood level for a second longer time period.

77. A sustained release kit according to claim 75, wherein each mini-implant includes
a pharmaceutical active-containing inner layer; and
a water-impermeable outer layer.

78. A sustained release kit according to claim 75, wherein the multiple sustained release mini-implants are packaged in a biodegradable sheath.

79. A sustained release kit according to claim 75, further including a delivery apparatus including an injector instrument for subcutaneous or intramuscular delivery of implants.

80. A method for the therapeutic or prophylactic treatment of a disease condition in an animal (including a human) requiring such treatment, which method includes administering to the animal a sustained release delivery apparatus including a plurality of sustained release mini-implants or pellets;
each mini-implant including
a pharmaceutically active composition including
at least one pharmaceutically active component; and
a carrier therefor; and
a sustained release support material, the pharmaceutically active composition being carried in or on the sustained release support material;
each implant being of insufficient size individually to provide a predetermined desired threshold blood level of pharmaceutical active for treatment of a selected disease indication;
the size(s) of the mini-implants or pellets providing zero order release of pharmaceutical active.

81. A method according to claim 80, wherein the mini-implants or pellets are provided in a first size which provides a blood level of pharmaceutical active of approximately 1.25 to 3 times the desired threshold blood level for a first relatively short time period; and
in a second size which provides a blood level of pharmaceutical active at or near the desired threshold blood level for a second longer time period.
